(19) United States
(12) Patent Application Publication
(10) Pub. No.: US 2003/0036539 A1
(43) Pub. Date: Feb. 20, 2003

(54) Use of 2-ME in promotion of apoptosis in the prevention of initial onset and recurrence of existing cancers

(75) Inventors: Thomas J. Slaga, Golden, CO (US); Addanki P. Kumar, Denver, CO (US)

Correspondence Address: DAVID G. HENRY, 900 Washington Avenue, P.O. Box 1470, Waco, TX, 76701, US

(73) Assignee: Oncology Sciences Corporation, Austin, TX

(21) Appl. No.: 10/210,778

(22) Filed: Aug. 1, 2002

Related U.S. Application Data
(63) Continuation of application No. 09/808,408, filed on Mar. 14, 2001.
Application No. 09/808,408 is a continuation-in-part of application No. 09/777,151, filed on Feb. 5, 2001.
Application No. 09/777,151 is a continuation-in-part of application No. 09/527,283, filed on Mar. 17, 2000, now abandoned.

(51) Int. Cl. A61K 31/56

(52) U.S. Cl. 514/182

(57) ABSTRACT

The use of 2-methoxyestradiol in the prevention of initial onset cancers and the recurrence of previously existing cancers, through use of such compound in a manner to promoteo pro-apoptotic activities in target cells and tissues.

### CITATION TO PRIOR APPLICATION(S)

This is a continuation application with respect to co-pending U.S. application Ser. No. 09/808,408, which was a continuation-in-part of U.S. application Ser. No. 09/777,151, which was a continuation-in-part with respect to U.S. application Ser. No. 09/527,283. Priority under 35 U.S.C. 120 is claimed for the instant application vis a vis the cited parent applications. This application discloses nothing other than as previously disclosed in the prior applications, but is filed to expedite consideration of the appended claims, in view of file processing irregularities in connection with the earliest priority application and its PCT counterpart, none of which have received final consideration.

### BACKGROUND OF THE INVENTION

A. Field of the Invention

The present invention relates to non-surgical intervention of cancers, including prostate cancer, as well as prophylactic use of herein identified compounds in the prevention of cancers.

B. Background of the Invention

Cancer is the second leading cause of death in the United States, accounting for approximately one in four deaths. Recent estimates by the American Cancer Society suggest that in excess of 500,000 people die from cancer every year—that is approximately 1,500 deaths a day. Further, approximately 2.5 million new cases of cancer were expected to be diagnosed in the year 2000 alone. At an estimated annual cost of $107 billion dollars in health care costs and lost productivity due to death and illness, cancer inflicts a vast human and monetary toll on the United States.

The generic use of the term “cancer” only hints at the vast diversity of anatomical structures that this disease affects and the myriad of molecular bases that form the foundation of this disease. The collective use of the word cancer includes diseases affecting the brain, breast, cervix uteri, colon, corpus uteri, kidney, renal pelvis, larynx, lung, bone marrow, bronchus, skin, lymph system, nervous system, oral cavity, pharynx, ovary, pancreas, prostate, rectum, stomach, testis, thyroid, urinary bladder, and others. The individual molecular bases of these diverse afflictions can be varied and diverse. However, among this diverse field of afflictions, there exist two unified modalities of cell growth and/or proliferation that are common to almost all types of cancer: 1) unchecked cell growth and/or immortality, and 2) angiogenesis.

One of the problems that characterize a vast number of cancers is the unregulated growth or unchecked life span of aberrant cells in the various tissues of the body. Normal cells grow, divide, and die on a regular basis. The process by which cells normally die is called apoptosis. However, when normal cell growth and death become unchecked in the body, by any number of processes, such unchecked growth and/or immortality leads to destroy the regular functioning of the various tissues of the body. Such growth or immortality can ultimately lead to the occurrence of a host of solid tumors, leukemia's, lymphomas, or the metastasis of cancer cells throughout the body. Unchecked cell growth and/or immortality are problematic biological mechanisms common to almost all types of cancer.

Another biological mechanism that is common to, and problematic in the prevention or treatment of, all solid cancer tumors is angiogenesis. Angiogenesis refers to the process by which new blood vessels are formed in the body. Without a dedicated blood supply, solid tumors have only limited growth potential—perhaps 2 mm in diameter maximum. However, angiogenesis often occurs in cancerous tissues and tumors, thus enabling solid tumors to sequester greater amounts of nutrients from the body and allowing them to proliferate rapidly, even spreading to other parts of the body. Angiogenesis is a critical means by which solid tumors grow rapidly and metastasize, hastening the process of death or disfigurement.

These two independent biological mechanisms are the common, primary modalities by which almost all cancer cells proliferate and grow. Hence, a novel approach for the treatment of cancer would be the development of pharmacological agents that have dual roles as anti-angiogenic as well as pro-apoptotic agents. Such an agent will have the ability to target both components of a cancer: kill the tumor cell by induction of apoptosis and cut off the blood supply to the tumor cell so that it will not grow.

In addition to the dire need for effective treatment modalities for existing cancers, there is arguably an even greater need for effective cancer preventative means, both with respect to initial onset of cancers, as well as in the context of recurrence of cancers after operative intervention.

### SUMMARY OF THE INVENTION

It is an object of the present invention to provide an agent that is efficacious in inhibiting the incidence, proliferation and/or angiogenesis of cancer cells.

It is another object of the present invention to provide a composition the primary active ingredient of which is 2-methoxyestradiol which is efficacious in inhibiting the incidence, proliferation and/or angiogenesis of cancer cells.

It is another object of the present invention to provide a method for inhibiting the proliferation and/or angiogenesis of cancer cells through use of a composition the primary active ingredient of which is 2-methoxyestradiol or an analogue thereof, as described herein.

It is, therefore, an object of the present invention to provide a new modality for the prevention of cancers as well as the suppression of recurrence of cancers once treated.

It is another object of the present invention to provide a new modality for the prevention of cancer as well as the suppression of recurrence of cancers once treated.

It is another object of the present invention to provide a new modality for the prevention of prostate cancer as well as the suppression of recurrence of prostate cancer once treated.

It is another object of the present invention to provide a method by which the known substance of 2-ME may be employed in a new and unobvious manner in the prevention of initial onset and of recurrence of cancers, including prostate cancer.

It is another object of the present invention to provide a method by which the known substance of 2-ME may be employed in the prevention of initial onset and post-operative recurrence of cancers, including prostate cancer.

It is another object of the present invention to provide a method by which the known substance of 2-ME may be employed in the prevention of initial onset and post-operative recurrence of cancers, including prostate cancer, through promotion of apoptotic activities in target cells. A recent breakthrough in the treatment of cancer, treated in the prior applications of the present inventors, is the use of 2-methoxyoestradiol (hereinafter “2-ME”). 2-ME is an endogenous non-toxic metabolic byproduct of estrogens that is present in human urine and blood. (1) A potential role for 2-ME as a chemopreventive agent has been reported in the mammary and pancreatic models. (2) 2-ME has also been shown to inhibit endothelial cell proliferation implicating its potential role in angiogenesis. (3) In addition, apoptosis has been implicated as a mechanism for 2-ME's cytostatic and anti-angiogenic effect.

2-methoxyestradiol (2-ME) is a metabolic byproduct of estrogens and an antimitotic agent that is present in human urine and blood. A potential role for 2-ME as a chemopreventive agent has been reported recently in the mammary and pancreatic models. 2-ME has been also shown to inhibit endothelial cell proliferation implicating its potential role in angiogenesis. These studies suggest a potential antitumorigenic role for 2-ME. In addition, 2-ME preferentially inhibits actively proliferating cells making it an ideal candidate for therapeutic applications. From these facts, the preset inventors postulated that 2-ME may protect men from prostate cancer, either by shrinking the tumors or by slowing their spread (metastasis) or by cutting the blood flow to the tumor (angiogenesis).

Therefore, in satisfaction of the above and related objects, the present invention provides both a composition and method for inhibiting the occurrence and proliferation of cancerous cells, and prostatic cells in particular. The composition is, and the method is based on the use of a composition consisting (among active ingredients) substantially of 2-methoxyestradiol. The present inventors have demonstrated beyond serious doubt that these compounds have a pronounced effect in inhibiting the incidence and proliferation of cancerous prostate cells and, therefore, provide a desperately needed stepping stone for advancing toward meaningful treatment of prostate and other cancers. Because of the mode of action (anti-tumorigenic, anti-angiogenic, and pro-apoptotic), 2-ME can be used, not only in the treatment of any kind of tumor, including prostate, brain, liver, lung, colon, and skin, but in preventing initial onset, or recurrence of such cancers after treatment.

Findings by the present inventors pertaining to the mechanisms of action of 2-ME serves as a cancer preventative agent, as well as curative agents. The present inventors previous work, filed with the original patent application and another continuation-in-part application, shows that 2-ME is of great significance in the treatment of prostate, brain, and nervous system cancer through the induction of apoptosis. This body of work indicates that 2-ME is an anti-tumorigenic agent with a significant therapeutic advantage since it can preferentially inhibit actively proliferating cells (characteristic of tumor cells) without affecting the growth of normal cycling cells. Additionally, 2-ME appears to also inhibit the formation of new blood vessels. To the best of our knowledge, this is the first compound that targets two components of cancer: the tumor cells and their blood supply. The present inventors have demonstrated that 2-ME is a chemical compound with a significant role as an antitumorigenic agent with broad efficacy in a variety of cancerous cell populations.

### DETAILED DESCRIPTION OF THE PREFERRED EMBODIMENT

The present inventors have tested the use of 2-ME in prostate cancer prevention in an in vitro system, and have determined that there exists proven efficacy in this regard. This testing has involved the use of androgen-dependent (LNCaP) and androgen-independent (DU145 and PC-3) cell lines to investigate the effect of 2-ME.

The cells were treated with different concentrations of 2-ME (0.5 to 5 μM) and cell growth, cell cycle progression and expression of p53 was monitored every 24 hours to understand the stage at which 2-ME acts on cancer cells.

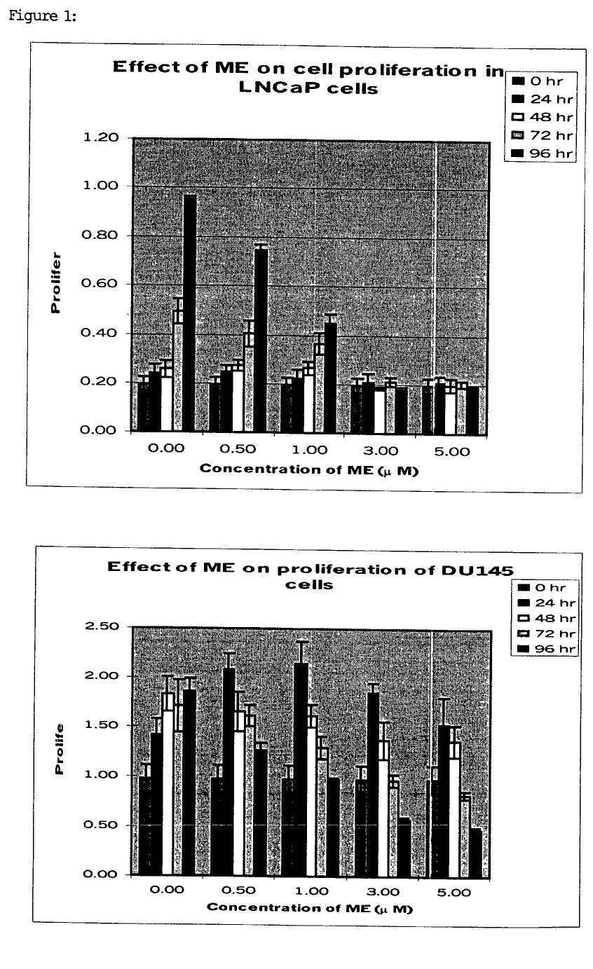

Referring to FIG. 1, actively growing LNCaP and DU145 cells were plated in 96-well plates at a density of 105 cells per well. After 24 hours in a 370C. incubator with 5% CO2, the cells were treated with the indicated concentration of 2-ME. Control cells received only the vehicle (DMSO). Cell growth was monitored every 24 hours using the CELLTITER96 AQUEOUS ONE solution assay containing a tetrazolium compound (Promega Corporation, Madison, Wis.).

The assay is based on the principle that actively growing cells generate reducing equivalents such as NADH that is necessary for the cells to reduce the tetrazolium compound to formazan product that is detected by measuring the absorbance at 570 nm. Increase in absorbance indicates cell viability.

The data reflected in FIG. 1 represents an average of five replicates and the experiment was conducted twice. As shown in FIG. 1, the control cells continued to proliferate during the time course of the experiment, the cells treated with 2-ME showed a dose-dependent inhibition of cell proliferation. The androgen-dependent LNCaP cell line was found to be more sensitive to the effect of 2-ME than the androgen-independent DU145 cell line. It is known that there are differences of AR and p53 between these two cell types.

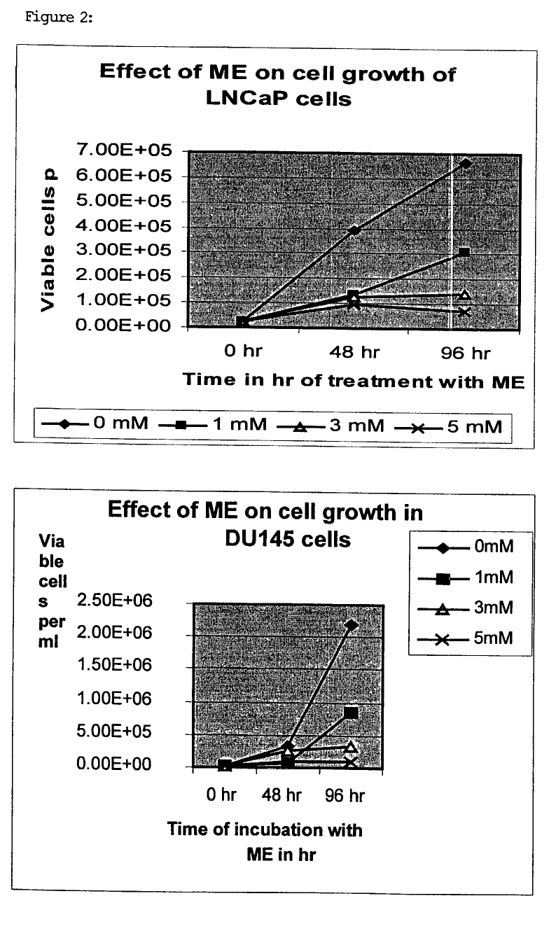

Referring to FIG. 2, the above experiment was confirmed by measuring cell viability using the trypan blue stain. LNCaP cells were grown in RPMI 1640 medium; and DU145 cells were grown in MEM Earles medium containing 10% Fetal bovine serum and penicillin and streptomycin (Life Technologies, Inc. Baltimore). Cells were plated at a density of 105 cells per 24 mm dish and after 24 hours, cells were treated with the indicated concentrations of 2-ME. Cell growth was monitored by harvesting the cells at the indicated time intervals following treatment with 2-ME using trypan blue exclusion method. The cell pellet was re-suspended in 0.5 ml of 0.4% trypan blue solution and after 15 minutes of incubation at RT, viable cells (unstained) were counted using a hemacytometer. The data reflected in FIG. 2 represents an average of three replicate dishes.

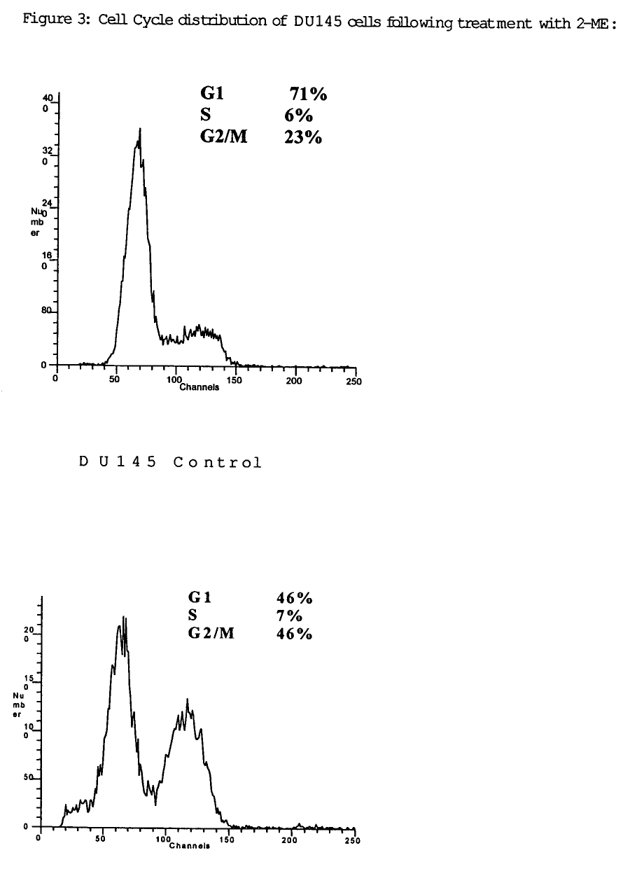

Referring to FIG. 3, since 2-ME inhibited the growth of LNCaP and DU145 cells, the present inventors set out to examine whether 2-ME treatment altered the distribution of cells during cell cycle progression. Logarithmically growing DU145 cells were plated in 60 mm dishes as described above at a density of 105 cells per dish. After 24 hours of growth at 370C. in an incubator with 5% CO2, half the dishes were treated with 3 μM of 2-ME and the other half was left untreated. Cells were observed every 24 hours for morphological changes following treatment with 2-ME. After 16 hours of incubation cells were harvested by trypsinization and the cell pellet was resuspended in 1 ml of Krishan stain and subjected to flow cytometric analysis at the Flow Cytometry facility of the University of Colorado Comprehensive Cancer Center, Denver, Colo. Flow cytometric analysis of the DU145 cells treated with 2-ME showed an increase in the G2/M population from 23% to 46% following treatment and a decrease in G1 population from 71% to 46% with no significant change in the population of cells in S phase. This data suggests that 2-ME inhibits growth of DU145 cells by arresting the cells predominantly in the G2/M phase. This could be due to alteration of expression and/or activities of cell cycle regulatory proteins in the G2/M phase.

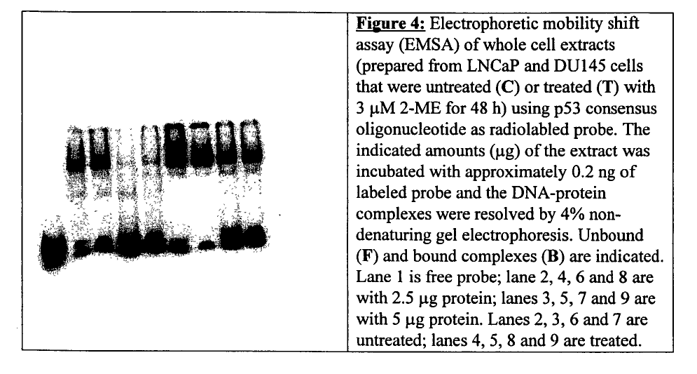

FIG. 4 depicts an electrophoretic mobility shift assay (EMSA) of whole cell extracts (prepared from LNCaP and DU145 cells that were untreated (C) or treated (T) with 3 μM 2-ME for 48 h) using p53 consensus oligonucleotide as radiolabled probe. The indicated amounts (mg) of the extract was incubated with approximately 0.2 ng of labeled probe and the DNA-protein complexes were resolved by 4% non-denaturing gel electrophoresis. Unbound (F) and bound complexes (B) are indicated. Lane 1 is free probe; lane 2, 4, 6 and 8 are with 2.5 μg protein; lanes 3, 5, 7 and 9 are with 5 μg protein. Lanes 2, 3, 6 and 7 are untreated; lanes 4, 5, 8 and 9 are treated.

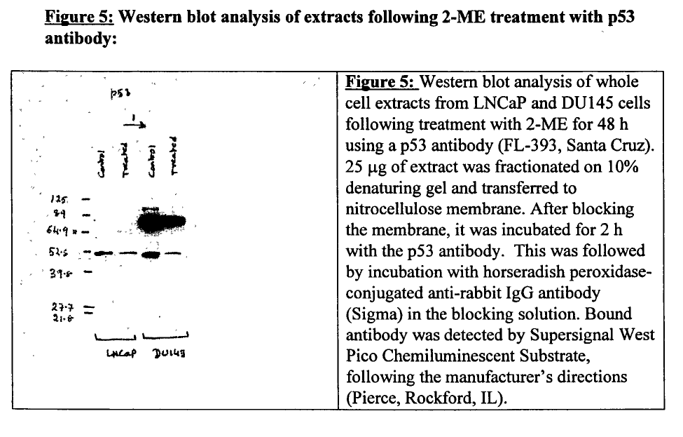

FIG. 5 depicts a Western blot analysis of whole cell extracts from LNCaP and DU145 cells following treatment with 2-ME for 48 hours using a p53 antibody (FL-393, Santa Cruz). 25 μg of extract was fractionated on 10% denaturing gel and transferred to nitrocellulose membrane. After blocking the membrane, it was incubated for 2 hours with the p53 antibody. This was followed by incubation with horseradish peroxidase-conjugated anti-rabbit IgG antibody (Sigma) in the blocking solution. Bound antibody was detected by Supersignal West Pico Chemiluminescent Substrate, following the manufacturer's directions (Pierce, Rockford, Ill.).

Referring to FIGS. 4 and 5, it is well established that the tumor suppressor function of p53 is mediated by accumulation of wild type p53 in response to extracellular signals with sequential induction of either cell cycle arrest or apoptosis. Mutation of p53 which is very frequent in human cancers (50%) is the result of disruption of these signaling pathways. Deregulation of such signaling pathways ultimately provides a selective growth advantage to tumor cells. Accordingly, the present inventors tested whether 2-ME induced growth inhibition was mediated by alterations in the DNA-binding activity of p53, its levels and/or its post-translational modifications.

As shown in FIG. 4 (Electrophoretic mobility shift assay (EMSA) of extracts prepared from LNCaP and DU145 cells following treatment with 2-ME using p53 oligonucleotide probe) and FIG. 5 (Western blot analysis of extracts following 2-ME treatment with p53 antibody), the proteins from LNCaP and DU145 cells bound to the p53 consensus sequence (FIG. 4, lanes 2 and 3). Interestingly, the proteins lost their binding activity following 2-ME treatment in LNCaP extracts (FIG. 4, lanes 4 and 5) which correlates with decrease in its expression (FIG. 5, lane 2). However, the loss of DNA-binding activity of DU145 extract was not as dramatic as that of LNCaP extracts. One would expect to see induced expression of p53 following 2-ME treatment, if it is involved in mediating apoptosis.

The use of 2-ME as a chemopreventive agent and/or chemotherapeutic agent offers the following important advantages: 1) it specifically targets (inhibits the growth) actively proliferating cells (characteristic feature of cancer cells) sparing the normal or slow growing cells thus increasing its therapeutic index; and 2) the fact that 2-ME inhibits angiogenesis suggests that it can be used in the treatment of any type of cancer since all types of cancers requires the growth of blood vessels (angiogenesis). Therefore limiting blood supply would limit the spread of cancerous cells to other tissues or organs (metastasis). Unlike androgen ablation therapy where recurrence of tumors occur due to development of androgen-independence, the use of 2-ME may be advantageous since 2-ME inhibits the growth of both androgen-dependent (LNCaP) and androgen-independent (DU145) cells. This may be an excellent choice for patients with metastatic prostate cancer. Furthermore, 2-ME in combination with other agents that are not cytotoxic such as silymarin, D-glucorate, selenium, vitamin D, phytic acid and other phytochemicals that work through different mechanisms may have a synergistic effect.

The present inventors have determined through similar experimentation to that described above that the following derivatives of 2-ME demonstrate similar efficacy to that of 2-ME when applied in the same manner as provided above for 2-ME:

1. 2-ethoxyestradiol;

2. 2-butoxyestradiol;

3. 17-α-ethynylestradiol with methoxy group at position 2;

4. 17-α-ethynylestradiol with butoxy group at position 2;

5. 17-α-ethynyl-9-α-fluoroestradiol with methoxy group at position 2; and

6. 17-α-ethynyl-9-α-fluoroestradiol with butoxy group at position 2.

Based on the above findings, it is anticipated that actual human treatment regimens will involve injection of 2-ME solutions directly into prostatic or other cancerous tumors, and systemic injections and ingestion (in tablet or capsule form) to effect a blood level suitable for inhibiting cancer cell proliferation.

Even before protocols for human use are developed, the present invention has utility, and provided above is an enabling disclosure in view of the proven inhibition of cancer cell proliferation though use of 2-ME will provide the foundation for further research and development in this field—itself of utilitarian value to the medical community.

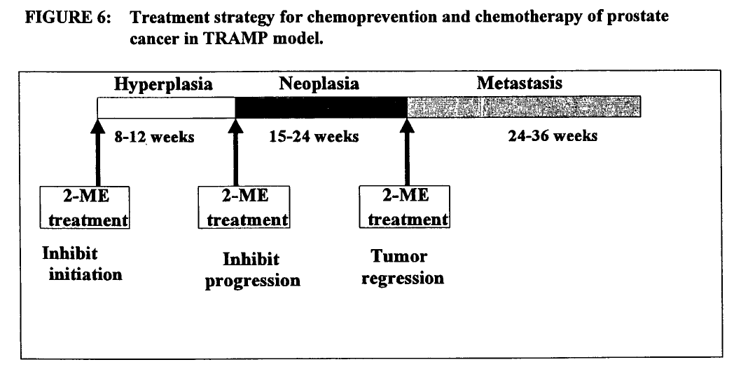

A treatment schedule, based on the progressive state of prostate cancer is suggested in FIG. 6. Treatment regimens for the prevention or treatment of other cancers may be extrapolated from the above.

Although the invention has been described with reference to specific embodiments, this description is not meant to be construed in a limited sense. Various modifications of the disclosed embodiments, as well as alternative embodiments of the inventions will become apparent to persons skilled in the art upon the reference to the description of the invention. It is, therefore, contemplated that the appended claims will cover such modifications that fall within the scope of the invention.

We claim:

1. A method of inducing apoptosis in cancerous tissues comprising the steps of:
administering a therapeutic dosage of a composition containing 2-methoxyestradiol to a cancerous tissues, said administration continuing at least until the initiation of cell apoptosis in said cancerous tissues.

2. A method for arresting growth of cancer tissues comprising the steps of:
administering a therapeutic dosage of a composition containing 2-methoxyestradiol to a cancerous tissue, said administration occurring at a time which, at least for some cells in said cancerous tissue, precedes cell division in the G2/M phase. m:

3. The use of compositions useful in the inhibition of cancerous cell genesis and proliferation, the primary activie ingredient of which is 2-methyloxyestradiol. said 2-methyloxyestradiol being administered in a therapeutic dosage at a time relative to target cell populations as to promote therapeutic apoptosis.
